(19) United States
(12) Patent Application Publication
(10) Pub. No.: US 2006/0111786 A1
(43) Pub. Date: May 25, 2006

(54) Metallic prosthetic implant for use in minimally invasive acromio-clavicular shoulder joint hemi-arthroplasty

(75) Inventor: David A. Petersen, Clearwater, FL (US)

Correspondence Address: LARSON AND LARSON, 11199 69TH STREET NORTH, LARGO, FL, 33773, US

(73) Assignee: ORTHOPEDIC DEVELOPMENT CORPORATION

(21) Appl. No.: 11/177,466

(22) Filed: Jul. 8, 2005

Related U.S. Application Data
(63) Continuation-in-part of application No. 10/992,697, filed on Nov. 22, 2004, now abandoned.

(51) Int. Cl. A61F 2/40

(52) U.S. Cl. 623/19.11

(57) ABSTRACT

A biocompatible metallic implant apparatus shaped to fit a natural contour of a shoulder joint, the implant having a cobalt-chrome convex outer surface, a porous concave inner surface and blades on the inner surface for insertion into bone. A locking screw through the implant adheres the implant to the shoulder joint.

### PRIOR APPLICATIONS

This application is a continuation-in-part from application SER. No. 10/992,697, filed Nov. 22, 2004.

### BACKGROUND OF THE INVENTION

The present invention relates to a metallic prosthetic implant for us in minimally invasive orthopedic acromio-clavicular joint surgery. More particularly, it refers to a minimally invasive arthroscopic type portal or classic open exposure joint hemi-arthroplasty for the right and left acromio-clavicular (hereinafter “a-c”) joints of the shoulder. The surgery employs the metallic prosthetic implant of this invention to resurface the a-c articulated joints using minimally invasive or arthroscopic portal type techniques to prepare the joint for such implantation.

Present surgical solutions available for the millions of people with small joint injuries, disease, degeneration and related dysfunctions are complex. Invasive operations, often requiring resection, are the only alternatives. Such operations greatly alter joint mechanics. In general, the present prior art requires prolonged recovery times and offers uncertain outcomes with high probability of persistent pain and disability.

For example, there were approximately 100,000 Mumford Procedures (acromio-clavicular shoulder joint resections) in the United States in 2003 that were candidates for minimally invasive a-c joint hemi-arthroplasty as an alternative. A destructive salvage procedure such as a Mumford Procedure results in greatly altered joint mechanics and, in a large number of patients, continued complaints of pain. An improved surgical implant is needed to minimize alteration of joint mechanics.

### SUMMARY OF THE INVENTION

The use of a pre-shaped metallic overlay of this invention for a-c joint resurfacing offers three distinct advantages over prosthetic implants which are presently used in small joint resection procedures. These advantages are: (1) using a thin metallic overlay allowing for minimally invasive insertion that is safer, less traumatic and requiring far less recovery time compared to resection; (2) the overlay minimizes the use of destructive methods or other fixation methods that can result in persistent pain over time; and, (3) the implant has one or more fins to provide lateral stability and one or more teeth to provide temporary fixation and a rough or porous inner surface amenable to bone in growth providing permanent natural fixation. The implant also has a polished outside that allows for smooth, natural, pain free articulation of the joint. It also utilizes a dynamic compression screw to provide immediate fixation.

The implant of this invention is specifically designed for use in an arthroscopic type portal or minimal incision open surgery and provides a unique, stronger and superior resurfacing significantly reducing the risk of collateral post-operative joint pain resulting from resection of the joint and all or part of the bone structure with a metallic or synthetic bone and joint.

The implant of this invention is used in painful, diseased or deteriorated right or left a-c joints of the shoulder.

The implant is a small metal on bone overlay. The overlay is constructed of a highly polished cobalt-chrome alloy, titanium alloy or such other biocompatible metal or metallic alloy appropriate for small joint hemi-arthroplasties. The implant is sized based on patient anatomy. The prosthesis is attached to the joint using a single screw. The amenable joint is accessed using an arthroscopic type portal or classic open surgery. Because the side that attaches to bone is rough or porous, the bone heals onto it, permanently fixing it into place. A uniquely designed set of blades and a single dynamic compression screw provides temporary fixation to the joint. The side making contact with the joint is highly polished providing a smooth, virtually frictionless surface that undergoes virtually no wear and tear.

The metallic overlay is generally shaped to the natural contour of the bone it resurfaces and is highly polished on the outside to provide frictionless articulation of the joint and is rough and porous on the inside to promote and provide a surface to allow the natural bone to grow into the overlay, providing a permanent fixation. In the interim between implantation and bone in-growth, the overlay is held in place by the dynamic compression screw.

### BRIEF DESCRIPTION OF THE DRAWINGS

Many advantages of the present invention will be apparent to those skilled in the art with a reading of this specification in conjunction with the attached drawings. Like reference numerals are applied to like elements wherein:

FIG. 1 is a top right isometric view of the prosthetic implant of the present invention.

FIG. 2 is a bottom left isometric view of the prosthetic implant of this invention.

FIG. 3 is a section view along lines 3-3 FIG. 1.

FIG. 4 is a sectional view similar to FIG. 3 with the patient's shoulder shown with a loose screw.

FIG. 5 is a sectional view of a patient's shoulder with an embedded insert and the screw tightened.

### DETAILED DESCRIPTION OF THE INVENTION

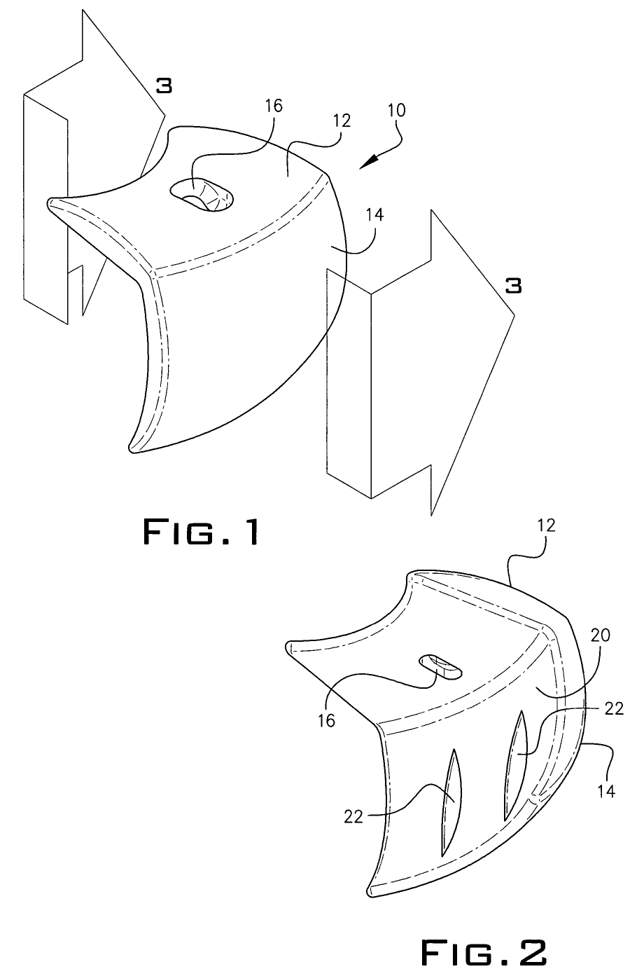

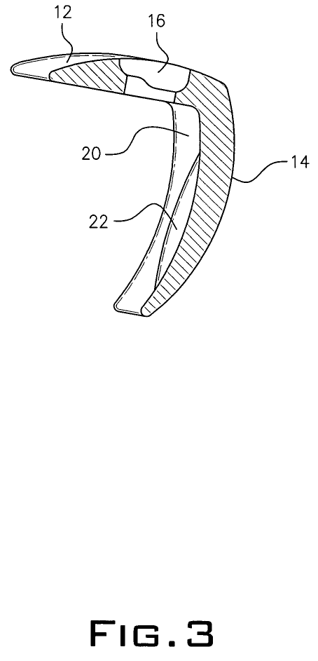

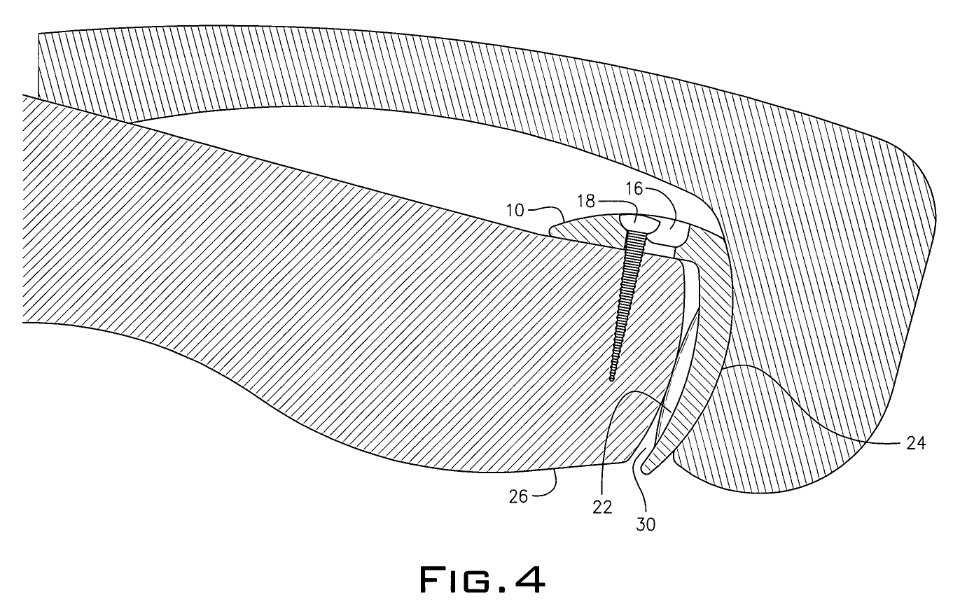

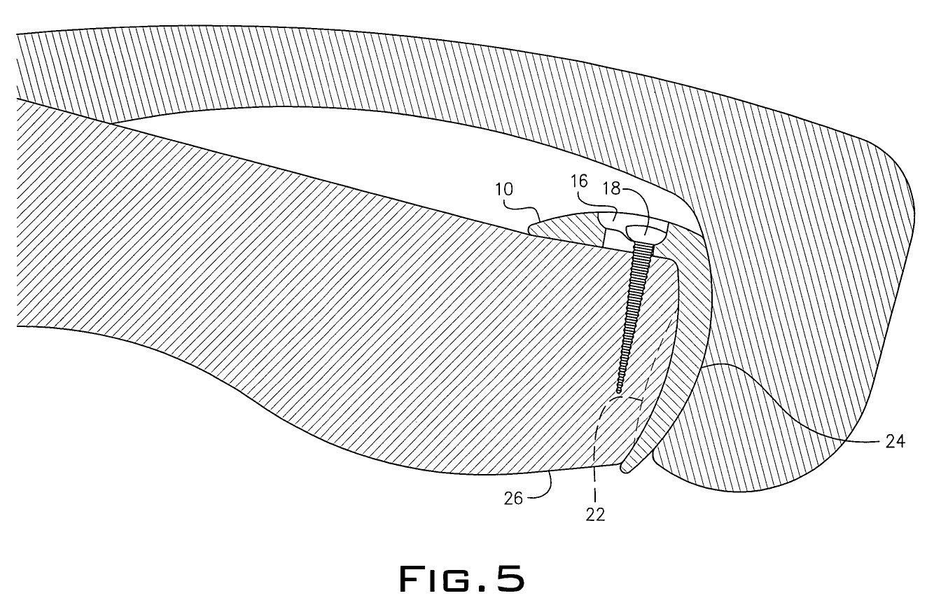

The minimally invasive a-c joint hemi-arthroplasty implant device 10 disclosed herein is shown in FIGS. 1 and 2. The implant 10 appears to look like an inverted “L” with convex top 12 and side 14 portion. The exterior surface of the top 12 and side 14 portion is a highly polished cobalt-chrome alloy, titanium alloy or other biocompatible material that promotes ease of contact in a patient's joint. A hole 16 in the top portion 12 is provided for entry of a locking screw 18 shown in FIGS. 4 and 5. The inner surface 20 of implant 10 forms a concave structure of the top 12 and side portion 14. A pair of blades 22 are attached to the inner surface 20 of the side portion 14.

Referring to FIGS. 4 and 5, implant 10 is inserted into a patient's damaged joint 24 so that the highly polished exterior surface of side 14 abuts the joint 24. The inside surface 20 of side portion 14 cuts into bone 26 with blade 22. The screw 18 is inserted into hole 16 and screwed into bone 26. As the screw 18 is tightened, the space 30 in the joint 24 closes down so the implant 10 is firmly located in joint 24. The inner surface 20 of side portion 14 is textured or porous to promote adhesion with bone 26.

Minimally invasive a-c joint hemi-arthroplasty solves the prior art problems by performing the procedure using an arthroscopic type portal or during a classic open exposure using a small resurfacing implant 10. Instead of a salvage procedure, this invention resurfaces the joint preserving function with reduced rehabilitation and pain.

Referring to FIG. 1, the convex exterior surface of side portion 14, of the implant 10 is constructed of a material such as highly polished cobalt-chrome alloy, an FDA approved metal for hemi-arthroplasty, allowing for smooth motion at the joint interface. The concave inner surface 20 of the implant 10 has a porous coating and fins or blades 22 that cut into the abraided bone 26 to provide for bone ingrowth. The locking screw 18 provides immediate secure fixation to allow for rapid rehabilitation.

Minimally invasive a-c joint hemi-arthroplasty is low risk, fast (about 15-20 minutes per joint in an outpatient setting), and has a recovery time estimated to be several weeks instead of months; and, it promises a high success rate, does not preclude other surgical options, is non-limiting and permanent.

The present invention overcomes, or at least improves upon, the disadvantages of the prior art by achieving the following:
Reversal of the cost/benefit ratio of the present procedures versus the invention;
A resurfacing procedure versus a salvage procedure;
A 15-20 minute outpatient procedure;
Reduced morbidity, blood loss, risk of infection resulting from reduced operating room time and tissue damage;
Recovery time significantly reduced;
No preclusion of other surgical or non-invasive treatment options; and,
Projected high success rate by utilizing accepted procedures facilitated through a new arthroscopic technique and resurfacing implant.

It is anticipated that the availability of implant 10 will increase the number of surgeries performed because it offers a safe outpatient surgical solution to a common problem. It is expected that virtually all patients receiving this implant 10 will be fully functional within a few weeks. Many of the present surgical solutions require months of painful recovery and therapy.

Aside from the obvious positive clinical outcome, the significant favorable financial impact on disability, worker's compensation and health care insurers is considerable.

The present inventi on accomplishes this goal by providing implant 10 to replace any of the right and left acromio-clavicular joints of the shoulder. The implant 10 will be appropriately sized for each of the patients. The joint 24 is accessed using a small incision or anthroscopic type portal eliminating the need for open salvage surgery and long recovery periods. Because the inside surface 20 of the implant 10 that attaches to bone is rough or porous bone in growth occurs, permanently fixing it into place. The locking screw 18 is applied to provide immediate fixation, holding the resurfacing implant 10 firmly until bone in growth is completed. The side 14 making contact with the joint 24 is highly polished providing a smooth, virtually frictionless surface that undergoes very little wear and tear. The device includes one or more fins or blades 22 to provide temporary lateral fixation.

By way of example, FIGS. 4 and 5 illustrate the device 10 in situ in an acromio-clavicular shoulder joint 24. The outer surface of the device 10 is polished presenting a metal to bone, virtually frictionless surface. One or more blades 22 on the inner concave porous contact surface 20 provides lateral fixation pending bone in growth. Additional temporary fixation is provided by a small screw 18.

Any number of instruments allow for preparation of the joint 24 and the implant 10 to be placed therein using a minimally invasive surgical arthroscopic technique by using a small incision to access to the joint. The instruments include those available in a well equipped operating room. A saw is used to make a small cut in the bone to prepare the surface for the implant. The locking screw 18 is used to fix the implant to prevent movement or migration prior to healing. Classic AO technique is used to compress the implant to the bone surface.

Equivalent features of the implants can be substituted for the features described for substantially the same function to produce substantially the same result in substantially the same way.

Having thus described the invention what is claimed and desired to be secured by Letters Patent is:

1. A biocompatible metallic implant shaped to fit a natural contour of an acromio-clavicular joint of a shoulder, the implant comprising:
a convex highly polished outer surface and a concave porous inner surface;
at least one blade on the inner surface for insertion into bone; and
a screw through a top surface of the implant for assistance in embedding the implant in the joint, the implant adapted for insertion through a minimally invasive anthroscopic type incision.

2. The biocompatible metallic implant according to claim 1, wherein the screw is a locking screw positioned through the outer surface of the implant and within an adjacent bone.

3. The biocompatible metallic implant according to claim 1, wherein the highly polished outer surface is a cobalt-chrome alloy.

4. The biocompatible metallic implant according to claim 1, wherein the highly polished outer surface is a titanium alloy.

5. The biocompatible metallic implant according to claim 1, positioned within an acromio-clavicular joint of a shoulder.

6. The biocompatible metallic implant according to claim 1, wherein there is a pair of blades on the porous inner surface for insertion into bone.

7. A biocompatible metallic implant shaped to fit a natural contour of an acromio-clavicular joint of a shoulder, the implant comprising:
a convex highly polished outer surface and a concave porous inner surface of a substantially L-shaped metallic body;
a pair of blades on the inner surface for insertion into bone; and
a screw passing through the outer surface of the implant, the implant adapted for insertion through a minimally invasive orthoscopic type incision.

8. The biocompatible metallic implant according to claim 7, wherein the outer surface is a cobalt-chrome alloy.

9. The biocompatible metallic implant according to claim 7, wherein the outer surface is a titanium alloy.

10. The biocompatible metallic implant according to claim 7, attached to an acromio-clavicular joint of a shoulder with a locking screw drilled through the outer surface of the implant and into adjacent bone.

11. The biocompatible metallic implant according to claim 7, wherein the concave porous inner surface is adopted to promote bone growth with adjacent bone tissue.

12. A biocompatible metallic implant inserted into a natural contour of an acrimio-clavicular joint of a shoulder, the implant comprising:
a convex brightly polished outer surface in contact with the joint and a concave porous inner surface in contact with bone;
at least one blade on the inner surface inserted into the bone; and
a screw passing through an outer surface of the implant and into bone, the implant inserted in place through an arthroscopic type incision.

13. A biocompatible metallic implant inserted into a natural contour of a joint according to claim 12, wherein the highly polished outer surface is a cobalt-chrome alloy.

14. A biocompatible metallic implant inserted in a natural contour of a joint according to claim 12, wherein there is a pair of blades on the porous unit surface inserted in bone.

15. A biocompatible metallic implant inserted into a natural contour of a joint according to claim 12, wherein the highly polished outer surface is a titanium alloy.
